(19) United States
(12) Patent Application Publication
(10) Pub. No.: US 2014/0236051 A1
(43) Pub. Date: Aug. 21, 2014

(54) APPARATUS AND METHOD OF TRANSMITTING ULTRASOUND

(71) Applicant: Samsung Electronics Co., Ltd., Suwon-si (KR)

(72) Inventors: Sang-hyun KIM, Hwaseong-si (KR); Won-chul BANG, Seongnam-si (KR)

(73) Assignee: Samsung Electronics Co., Ltd., Suwon-si (KR)

(21) Appl. No.: 14/073,266

(22) Filed: Nov. 6, 2013

(30) Foreign Application Priority Data
Feb. 19, 2013 (KR) 10-2013-0017659

(51) Int. Cl. A61N 7/00

(52) U.S. Cl. CPC A61N 7/00
USPC 601/3

(57) ABSTRACT

Provided are methods and apparatuses of transmitting ultrasound by adjusting positions of a plurality of ultrasound generating elements within an applicator to direct the ultrasound waves at a position desired by a user.

### CROSS-REFERENCE TO RELATED APPLICATIONS

This application claims the benefit under 35 U.S.C. §119(a) of Korean Patent Application No. 10-2013-0017659, filed on Feb. 19, 2013, in the Korean Intellectual Property Office, the entire disclosure of which is hereby incorporated by reference for all purposes.

### BACKGROUND

1. Field

The following description relates to methods of transmitting ultrasound and ultrasound apparatuses using the same. The following description also relates to a method of transmitting ultrasound by adjusting positions of a plurality of ultrasound generating elements that comprise a transducer and an ultrasound apparatus using the same.

2. Description of Related Art

Ultrasound refers to an acoustic wave having frequencies above the human audible range. Ultrasound is used in various fields including the medical field. Ultrasound may be used to diagnose a disease by sending ultrasound waves to a tissue or an organ having a lesion within the human body, receiving a signal reflected from the tissue or organ, and obtaining an ultrasound image of the tissue or organ. In an ultrasound treatment, minimal invasive surgery and non-invasive surgery have been performed using ultrasound. As a non-invasive treatment method, a high intensity focused ultrasound (HIFU) treatment using ultrasound has been widely used as a treatment that is harmless to the human body. An HIFU treatment causes necrosis of the tissue of the lesion in the human body by focusing high intensity ultrasound on the tissue of the lesion. The HIFU treatment includes forming a burn by irradiating therapeutic ultrasound to the tissue of the lesion through a therapeutic ultrasound device when the tissue of the lesion is found in an object, acquiring ultrasound images of the tissue that includes the tissue of lesion through a diagnostic ultrasound device, and diagnosing whether treatment is completed.

A technique for focusing ultrasound on a position desired by a user or steering it toward a desired direction is significant in using an ultrasound apparatus. Thus, research has been actively carried out regarding steering and focusing of ultrasound beams.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided an ultrasound apparatus including: a transducer comprising a plurality of ultrasound generating elements controlled independent of each other and configured to emit ultrasound waves at positions adjusted according to calculated displacements; a plurality of actuators configured to adjust position of the plurality of ultrasound generating elements; a displacement calculator configured to calculate displacements of the plurality of ultrasound generating elements based on a position of an object to be irradiated with ultrasound waves; a displacement adjuster configured to control the plurality of actuators based on the calculated displacements; and a driver configured to drive the plurality of ultrasound generating elements.

The driver may be further configured to simultaneously generate drive signals for driving the plurality of ultrasound generating elements.

A focal point where the emitted ultrasound waves are focused may be determined by the positions of plurality of ultrasound generating elements.

Each of the ultrasound waves emitted by the plurality of ultrasound generating elements may have a different phase value due to a different position of each of the plurality of ultrasound generating elements.

The direction of an ultrasound beam generated by the transducer may be changed by adjusting the positions of the plurality of ultrasound generating elements.

The calculated displacement may be less than one wavelength of the emitted ultrasound wave.

The apparatus may include at least one movement support that is configured to support at least one of the plurality of actuators and to assist in movement of the at least one of the plurality of actuators, and a movement controller may be configured to move the at least one actuator within a predetermined range along the at least one movement support.

If the at least one actuator is moved along the at least one movement support, the displacement calculator may be further configured to calculate the displacements of the plurality of ultrasound generating elements within the applicator based on positions of the plurality of ultrasound generating elements that are moved together with the at least one actuator.

The transducer, the plurality of actuators, and the displacement adjuster may be disposed in an applicator.

The driver may be implemented in a single-channel architecture, and the driver may include a generator and a power amplifier.

In another general aspect, there is provided a method of transmitting ultrasound waves using a transducer disposed within an applicator, the transducer comprising a plurality of ultrasound generating elements controlled independently of each other through a plurality of actuators, the method including: calculating displacements of the plurality of ultrasound generating elements based on a position of an object to be irradiated with ultrasound waves; adjusting positions of the plurality of actuators based on the calculated displacements; generating drive signals for driving the plurality of ultrasound generating elements; and emitting the ultrasound waves at the positions adjusted according to the calculated displacements.

The generating of the drive signals may include generating the drive signals simultaneously.

A focal point where the emitted ultrasound waves are focused may be determined by the positions of plurality of ultrasound generating elements.

Each of the ultrasound waves emitted by the plurality of ultrasound generating elements may have a different phase value due to a different position of each of the plurality of ultrasound generating elements.

The direction of an ultrasound beam generated by the transducer may be changed by adjusting the positions of the plurality of ultrasound generating elements.

The calculated displacement may be less than one wavelength of the emitted ultrasound wave.

In another general aspect, there is provided an ultrasound applicator including: a housing with an open end; a membrane disposed on the open end of the applicator and the membrane configured to contact the skin of a subject; a transducer comprising a plurality of ultrasound generating elements controlled independent of each other, the transducer is disposed in the housing; a plurality of actuators configured to adjust position of the ultrasound generating elements, the actuators are disposed in the housing; and a displacement adjuster configured to control the plurality of actuators based on a position of the object to be irradiated with ultrasound waves, the displacement adjuster is disposed in the housing.

The transducer may be disposed proximal to the membrane, the displacement adjuster may be disposed proximal to a closed end of the housing, and the actuators may be disposed between the ultrasound generating elements and the displacement adjuster.

At least one movement support may be disposed in the housing, the movement support may be configured to support at least one of the plurality of actuators and to assist in movement of the at least one of the plurality of actuators.

The shape of the movement support may be linear, curved, or spiral.

Other features and aspects will be apparent from the following detailed description, the drawings, and the claims.

### BRIEF DESCRIPTION OF THE DRAWINGS

FIG. 1 is a diagram illustrating an example of a structure of an ultrasound system.

FIG. 2 is a diagram illustrating an example of components of an ultrasound apparatus in the ultrasound system of FIG. 1.

FIG. 3 is a diagram illustrating an example of an internal structure and operation of an applicator in the ultrasound apparatus.

FIGS. 4A, 4B, and 4C are diagrams illustrating examples of position of a focal point at which ultrasound waves are focused according to positions of ultrasound generating elements within an applicator.

FIG. 5 is a diagram illustrating an example of an internal construction and operation of an applicator in an ultrasound apparatus.

FIGS. 6A and 6B are diagrams illustrating examples of characteristics of an internal construction of a driver used in an ultrasound apparatus.

FIG. 7 is a diagram illustrating an example of a method of transmitting ultrasound.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

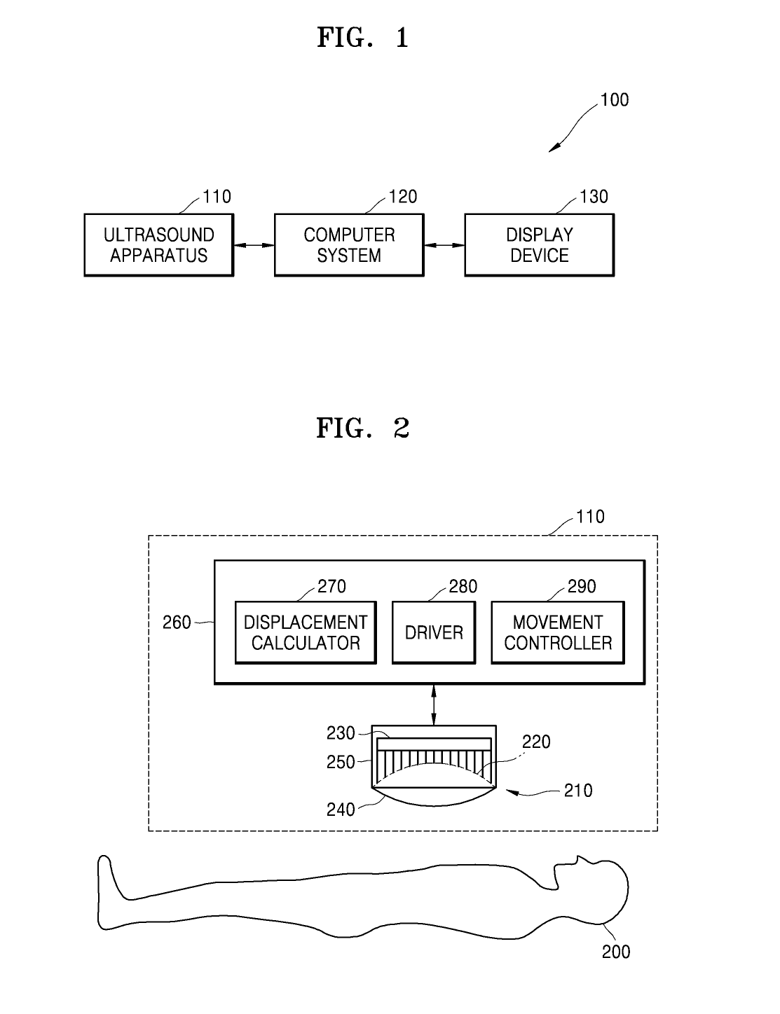

FIG. 1 is a diagram illustrating an example of a structure of an ultrasound system 100. Referring to FIG. 1, the ultrasound system 100 includes an ultrasound apparatus 110, a computer system 120, and a display device 130. Only components related to the present example are illustrated in the ultrasound system 100 of FIG. 1. Thus, those skilled in the art may understand that general components except for components illustrated in FIG. 1 may be further included. The individual elements in the ultrasound system 100 may not be physically separated from each other as shown in FIG. 1, but may be integrated into a single system.

When an abnormal area such as a tumor is found in a tissue, the ultrasound system 100 uses the ultrasound apparatus 110 for treatment by sending a therapeutic ultrasound wave to the abnormal area. The ultrasound system 100 generates and displays images of the tissue that has the abnormal area by receiving an echo signal reflected after irradiating the tissue with a diagnostic ultrasound wave. The displayed images may help medical practitioners to determine whether the abnormal area has been adequately treated.

The ultrasound apparatus 110 is used to treat a subject by sending ultrasound waves to an object such as abnormal tissue to cause necrosis of the abnormal tissue. A diagnostic probe for irradiating ultrasound waves for diagnosis may be integrated into or may be separated from an applicator of the ultrasound waves within the ultrasound apparatus 110. The diagnostic probe transmits ultrasound waves to the tissue that has an object, such as an abnormal area, and receives echo signal reflected from the tissue. The echo signal is used to generate an image of the object and/or the tissue. In addition to generating an ultrasound image, the echo signal may be used for monitoring changes in a temperature or status of the tissue that has the abnormal area. The ultrasound apparatus 110 will be described in more detail below with reference to FIG. 2.

The computer system 120 receives a command from a user, transmits data or signals required for operation of the ultrasound apparatus 110 to the ultrasound apparatus 110, and generates an image from the data received from the ultrasound apparatus 110. For example, the computer system 120 may generate an ultrasound image of tissue that has an object such as an abnormal area by using an echo signal received by the diagnostic probe in the ultrasound apparatus 110. For this purpose, the computer system 120 includes at least one image processor and a user interface for receiving a user command. The user interface may also be responsible for inputting and outputting input information regarding a user and an image. The interface unit may include a network module for connection to a network and a universal serial bus (USB) host module for forming a data transfer channel with a mobile storage medium, depending on a function of the ultrasound system 100. In addition, the user interface may include an input/output device such as, for example, a mouse, a keyboard, a touch screen, a monitor, a speaker, a screen, and a software module for running the input/output device.

The display device 130 receives a signal representing the ultrasound image from the computer system 120 and displays the ultrasound image on a display. The display device 130 may be implemented as a liquid crystal display (LCD), a light-emitting diode (LED) display, a plasma display panel (PDP), a screen, a terminal, and the like. A screen may be a physical structure that includes one or more hardware components that provide the ability to render a user interface and/or receive user input. The screen can encompass any combination of display region, gesture capture region, a touch sensitive display, and/or a configurable area. The screen can be embedded in the hardware or may be an external peripheral device that may be attached and detached from the apparatus. The display may be a single-screen or a multi-screen display. A single physical screen can include multiple displays that are managed as separate logical displays permitting different content to be displayed on separate displays although part of the same physical screen.

FIG. 2 is a diagram illustrating an example of components of the ultrasound apparatus 110 in the ultrasound system 100. Referring to FIG. 2, the ultrasound apparatus 110 includes an applicator 210 and a control unit 260. The applicator 210 includes a transducer 220, a displacement adjuster 230, a membrane 240, and a housing 250. The control unit 260 includes a displacement calculator 270, a driver 280, and a movement controller 290. Those skilled in the art may understand that the ultrasound apparatus 110 may further include commonly used components other than the components illustrated in FIG. 2.

The applicator 210 irradiates ultrasound waves. The applicator 210 comprises a housing 250 and the transducer 220 is disposed within the housing 250. The transducer 220 generates and transmits the ultrasound waves. The housing 250 has a bottom surface, which is attached to the membrane 240. The membrane 240 of the applicator 210 contacts a skin of a subject to send ultrasound waves into the body of the subject.

The transducer 220 generates ultrasound waves and transmits the ultrasound waves to an object. The object denotes abnormal tissue such as an abnormal area within a subject's body. The transducer 220 includes a plurality of ultrasound generating elements, each of which has a built-in piezo resonator for converting electrical energy into an ultrasound wave or vice versa. The ultrasound generating elements are controlled independently of each other. A plurality of drive signals may be input to the respective ultrasound generating elements, and positions of the plurality of ultrasound generating elements may be adjusted individually. The transducer 220 may consist of two or more ultrasound generating elements, which are arranged in a linear or curved array. The ultrasound generating elements may also be arranged as an n×m matrix, or may be closely spaced in the shape of a circle or curved surface. Alternatively, a plurality of separate transducers 220 may be configured to focus ultrasound waves at a single point.

The displacement adjuster 230 controls an actuator based on a displacement received from outside. The displacement is a vector having direction and magnitude that extends from a first position to a second position. Each of the ultrasound generating elements in the transducer 220 is coupled to an actuator (not shown) so that a position of each ultrasound generating element is individually adjusted. The actuator may comprise a motor that uses electricity, fluid pressure, compressed air, etc to adjust the position of the ultrasound generating element. The actuator described above is only a non-exhaustive illustration, and other types of actuators, which are configured to adjust the position of the ultrasound generating element are considered to be well within the scope of the present disclosure. The displacement adjuster 230 controls the actuator to adjust positions of the ultrasound generating elements mechanically. Since each actuator is combined with a corresponding ultrasound generating element, a position of each ultrasound generating element within an applicator 210 is independently adjusted by the actuator. The displacement adjuster 230 receives calculated displacements of the individual ultrasound generating elements within the applicator 210 from the displacement calculator 270 and controls the actuators corresponding to the individual ultrasound generating elements based on the displacements.

The membrane 240 is disposed in a direction that the transducer 220 irradiates ultrasound waves and creates a space where cooling liquid circulates. The membrane 240 may be made of an elastic material, which cannot be penetrated by the cooling liquid. The elastic material may increase the area of contact of the membrane 240 with a subject's skin, thereby creating a window through which the ultrasound waves can pass. One side of the membrane 240 is in contact with the subject's skin while the other side thereof is in contact with the cooling liquid.

The housing 250 encloses an outer surface of the applicator 210 to protect the internal components of the applicator 210 against external stimulation and to provide a space for accommodating the internal components of the applicator 210.

The control unit 260 controls the operations of the components of the ultrasound apparatus 110. The control unit 260 may generate control signals and control data for components of the applicator 210 and transmits the same to the applicator 210. The control unit 260 also collects data generated by the applicator 210 and transmits the collected data to the computer system 120 to generate an ultrasound image signal. The control unit 260 may be divided into several components according to their functions. According to a non-exhaustive example, the control unit 260 includes the displacement calculator 270, the driver 280, and the movement controller 290.

The displacement calculator 270 calculates displacements of the plurality of ultrasound generating elements within the applicator 210 based on the position of an object to be irradiated with ultrasound waves. The displacement calculator 270 calculates directions and distances from the current positions of each ultrasound generating element to an adjusted position such that a focal point is formed at which ultrasound waves are focused at the object. The displacement calculator 270 receives information about the positions of each ultrasound generating element and arrangements of the ultrasound generating elements according to the position of the focal point at which ultrasound waves are focused. The displacement calculator 270 may determine a displacement of each ultrasound generating element within the applicator 210 as a value that is less than one wavelength of an ultrasound wave. This is to prevent ultrasound generating elements from interfering with ultrasound irradiation of each other at an adjusted position.

The driver 280 drives the plurality of ultrasound generating elements that comprise the transducer 220. The driver 280 simultaneously generates drive signals for driving the plurality of ultrasound generating elements, amplifies the drive signals, and transmits the resultant drive signals to the ultrasound generating elements. Since the drive signals for driving the plurality of ultrasound generating elements are produced simultaneously, the driver 280 may be simplified into a single channel. If the driver 280 is configured to generate drive signals having a phase difference due to a time delay to drive each ultrasound generating element, the driver 280 requires a separate channel for each ultrasound generating element.

In addition to the displacement calculator 270 and the driver 280, the control unit 260 may further include the movement controller 290. As described above, the displacement adjuster 230 within the applicator 210 controls the actuators for controlling the positions of the ultrasound generating elements. A position of each ultrasound generating element coupled to a corresponding actuator is dependent on the specifications of the actuator such as a travel range of the actuator and a direction in which the actuator can move the ultrasound generating element. Thus, by moving the actuator that is combined with the corresponding ultrasound generating element within the applicator 210, limitations due to the travel range or movement direction of the actuator may be eliminated. To achieve this, the control unit 260 may further include the movement controller 290 that is configured to move the actuator within a predetermined range in the applicator 210.

The movement controller 290 may move at least one actuator within a predetermined range in the applicator 210 along a movement support (not shown) in the applicator 210. The movement support is a structure that supports at least one actuator in an interior space of the applicator 210 and assists in the movement of the actuator. For example, when an obstacle exists on a propagation path of an ultrasound beam, through which the ultrasound beam cannot pass, the movement controller 290 may move the actuators coupled with the ultrasound generating elements to avoid the obstacle, thus allowing irradiation with the ultrasound beam. When the movement controller 290 moves an actuator within a predetermined range in the applicator 210, the displacement calculator 270 calculates a displacement of a corresponding ultrasound generating element based on a position of the ultrasound generating element moving together with the actuator.

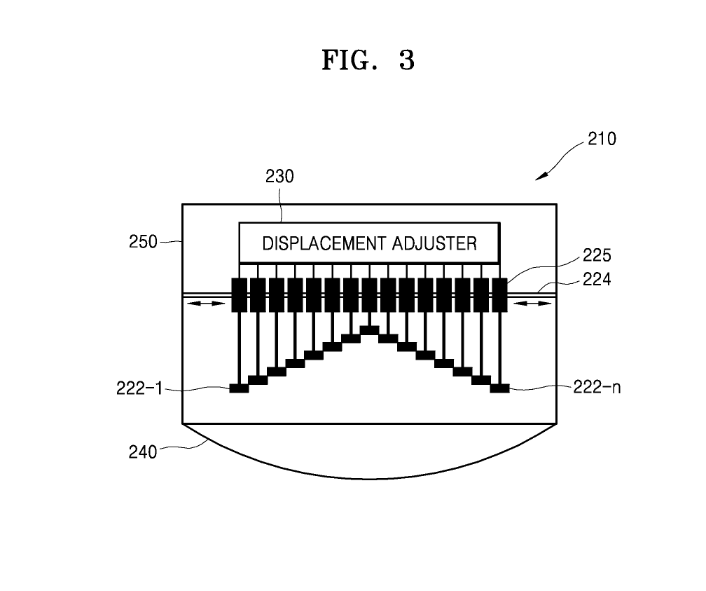

Operations within the ultrasound apparatus 110 will now be described in detail with reference to FIGS. 3 through 6 FIG. 3 is a diagram illustrating an example of an internal structure and operation of an applicator 210 in the ultrasound apparatus 110. Only components related to the present example are illustrated in the applicator 210 of FIG. 3. Thus, those skilled in the art may understand that general components except for components illustrated in FIG. 3 may be further included.

The applicator 210 includes a housing 250 and a membrane 240 on the outside. The membrane 240 is attached to a bottom side of the housing 250 and contacts a subject's skin. A plurality of ultrasound generating elements 222-1 through 222-n, which comprise a transducer, are disposed within the housing 250 of the applicator 210. The plurality of ultrasound generating elements 222-1 through 222-n in the transducer may be arranged in a in a linear or curved array. The ultrasound generating elements may also be arranged as an n×m matrix. FIG. 3 is a cross-section of the applicator 210 including a liner array transducer having n ultrasound generating elements 222-1 through 222-n. When the number of linear array transducers is m, the transducer may be configured into an n×m two-dimensional (2D) array.

One side of each of the plurality of ultrasound generating elements 222-1 through 222-n is coupled to one of a plurality of actuators 225. The plurality of actuators 225 are also connected to a displacement adjuster 230 and receive signals for controlling the actuators 225 from the displacement adjuster 230 to adjust the position of the plurality of ultrasound generating elements 222-1 through 222-n within the applicator 210. In a non-exhaustive example, the actuators 225 may mechanically adjust the positions of the ultrasound generating elements 222-1 through 222-n in response to the control signals. For example, the actuator 225 includes a cylindrical body and a piston reciprocating in the cylindrical body in response to an external electrical signal. One side of the piston is coupled to one side of the corresponding ultrasound generating element. Thus, as the piston moves, the position of the ultrasound generating element coupled to the piston is adjusted accordingly. Referring to FIG. 3, moving toward the center, the positions of the ultrasound generating elements 222-1 through 222-n are adjusted toward the actuators 225.

At least one movement support 224 may be disposed within the applicator 210, support at least one actuator 225, and assist in the movement of the actuator. One movement support 224 may be provided for each actuator 225, or the plurality of actuators 225 may be mounted on a single movement support 224. The number of movement supports 224 may be determined according to the shape of a transducer. For example, for a one-dimensional (1D) linear array transducer, one movement support 224 is provided for each actuator 225. For an n×m 2D linear array transducer, a number of n actuators 225 arranged in a line may be mounted together to the same movement support 224. The actuators 225 are movable within a predetermined range along the movement support 224. While the movement support 224 has a linear shape, the present disclosure is not limited thereto. The movement support 224 may have various shapes such as, for example, a curved shape and a spiral shape depending on the distribution of the actuators 225 and the ultrasound generating elements 222-1 through 222-n within the applicator 210.

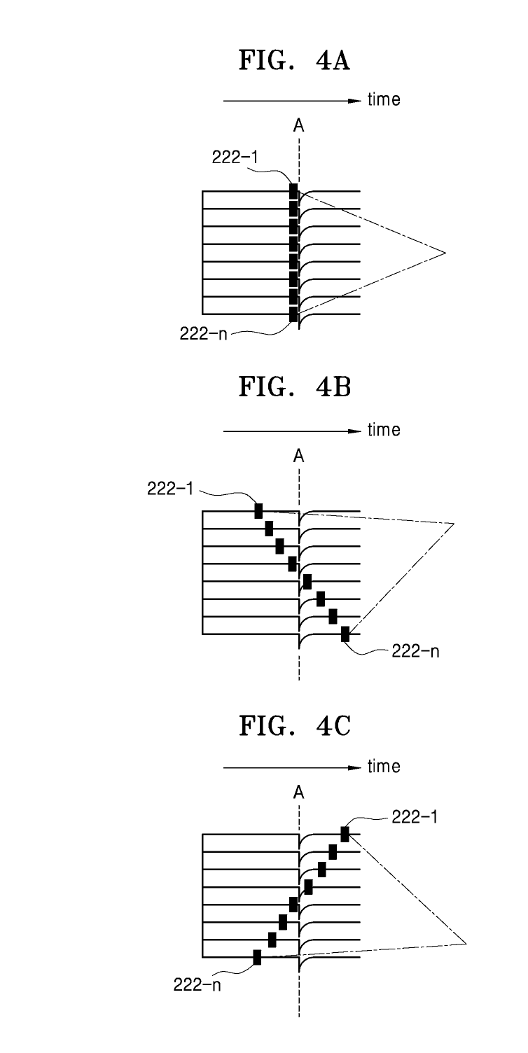

FIGS. 4A, 4B, and 4C are diagrams illustrating examples of positions of a focal point at which irradiated ultrasound waves are focused according to positions of the ultrasound generating elements 222-1 through 222-n within an applicator. FIGS. 4A, 4B, and 4C illustrate different arrangements of the ultrasound generating elements 222-1 through 222-n. In a non-exhaustive example and for convenience of explanation, the ultrasound generating elements 222-1 through 222-n are arranged to form a linear array transducer. However, other arrangements of the ultrasound generating elements 222-1 through 222-n are considered to be well within the scope of the present disclosure. As shown in FIG. 4, the drive signals for driving the individual ultrasound generating elements are generated and transmitted simultaneously, i.e., the drive signals are at the same position at point in time ‘A’. There is no delay between individual drive signals.

FIG. 4A illustrates an example of an arrangement of the ultrasound generating elements 222-1 through 222-n in a straight line. In this case, when drive signals for driving individual ultrasound generating elements 222-1 through 222-n reach the corresponding ultrasound generating elements 222-1 through 222-n at the same time, the ultrasound generating elements 222-1 through 222 simultaneously generate ultrasound waves for irradiation. A location where the greatest amount of overlap occurs between energies of the ultrasound waves becomes a focal point where the ultrasound waves are focused. As shown in FIG. 4A, a focal point is created in a front side of the ultrasound generating elements 222-1 through 222-n, the front side is the side that is distal from the actuators 225. The focal point is located at a central of the ultrasound generating elements 222-1 through 222-n.

FIG. 4B illustrates another example of an arrangement of the ultrasound generating elements 222-1 through 222-n along a diagonal line running from the upper left to the lower right of the line denoting the point in time ‘A’. In this non-exhaustive example, the drive signals for driving the individual ultrasound generating elements 222-1 through 222-n are generated simultaneously and transmitted at the same time as described with reference to FIG. 4A. However, the drive signals arrive at the corresponding ultrasound generating elements 222-1 through 222-n in a different order depending on the positions of the ultrasound generating elements 222-1 through 222-n. Thus, a drive signal arrives first at a first ultrasound generating element 222-1 on the upper left while a drive signal arrives last at an n-th ultrasound generating element 222-n on the lower right. The ultrasound generating elements 222-1 through 222-n are driven sequentially from the first ultrasound generating element 222-1 to the n-th ultrasound generating element 222-n. As shown in FIG. 4B, the location of a focal point and the direction of an ultrasound beam are changed toward the first ultrasound generating element 222-1, where the drive signal arrives first. This is different from FIG. 4A where the focal point is located at the center of the ultrasound generating elements 222-1 through 222-n.

FIG. 4C illustrates another example of an arrangement of the ultrasound generating elements 222-1 through 222-n along a diagonal line extending from the upper right to the lower left of the line denoting the point in time ‘A’. In this non-exhaustive example, the drive signals for driving the individual ultrasound generating elements 222-1 through 222-n are generated simultaneously and transmitted at the same time as described with reference to FIG. 4A. However, the drive signals arrive at the corresponding ultrasound generating elements 222-1 through 222-n in a different order depending on the positions of the ultrasound generating elements 222-1 through 222-n. Thus, a drive signal arrives last at a first ultrasound generating element 222-1 on the upper right while a drive signal arrives first at an n-th ultrasound generating element 222-n on the lower left. The ultrasound generating elements 222-1 through 222-n are driven sequentially from the n-th ultrasound generating element 222-n to the first ultrasound generating element 222-1. As shown in FIG. 4C, the location of a focal point and the direction of an ultrasound beam are changed toward the n-th ultrasound generating element 222-n, where at which the drive signal arrives first. This is different from FIG. 4A, where the focal point is located at the center of the ultrasound generating elements 222-1 through 222-n.

As can be seen from FIGS. 4A, 4B, and 4C, the position of a focal point at which ultrasound waves are focused or the direction of an ultrasound beam may vary according to the positions of the ultrasound generating elements 222-1 through 222-n within the applicator. The direction of an ultrasound beam generated by the transducer may be steered by adjusting the positions of the ultrasound generating elements 222-1 within the applicator. Each ultrasound wave emitted by each of the ultrasound generating elements 222-1 through 222-n has a different phase value due to a different position of each of the plurality of ultrasound generating elements 222-1 through 222-n within the applicator.

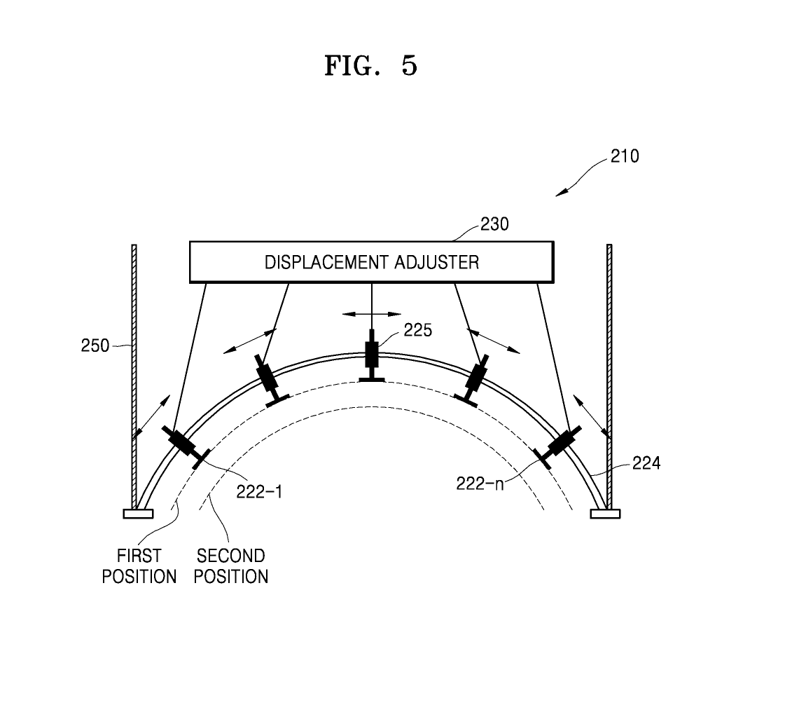

FIG. 5 is a diagram illustrating an example of an internal construction and operation of an applicator 210 in an ultrasound apparatus. Only components related to the present example are illustrated in the applicator 210 of FIG. 5. Thus, those skilled in the art may understand that general components except for components illustrated in FIG. 5 may be further included.

FIG. 5 is a cross-section of the applicator 210 including a curved array transducer having n ultrasound generating elements, 222-1 through 222-n. Referring to FIG. 5, a plurality of ultrasound generating elements 222-1 through 222-n, which comprise a transducer, are disposed within the housing 250 of the applicator 210. Unlike the transducer of FIG. 3, the plurality of ultrasound generating elements 222-1 through 222-n are arranged in a curved shape, or form a curved surface. A plurality of curved array transducers may be combined to form a curved surface transducer.

One side of each of the plurality of ultrasound generating elements 222-1 through 222-n is coupled to a corresponding actuator 225. The plurality of actuators 225 are also connected to a displacement adjuster 230 and receive signals for controlling the actuators 225 from the displacement adjuster 230 to thereby adjust positions of the plurality of ultrasound generating elements, 222-1 through 222-n, within the applicator 210. In a non-exhaustive example, the actuators 225 may mechanically adjust the positions of the ultrasound generating elements 222-1 through 222-n in response to the control signals. For example, the actuator 225 includes a cylindrical body and a piston reciprocating in the cylindrical body in response to an external electrical signal. One side of the piston is coupled to one side of the corresponding ultrasound generating element. Thus, as the piston moves, the position of the ultrasound generating element coupled to the piston is adjusted accordingly. Referring to FIG. 5, the actuator 225 operates so that the ultrasound generating elements 222-1 through 222-n may move from a first position to a second position. Due to the change in position of the ultrasound generating elements 222-1 through 222-n, the transducer including the ultrasound generating elements 222-1 through 222-n may have a smaller curved or curved surface shape.

At least one movement support 224 may be disposed within the applicator 210, support at least one actuator 225, and assist in the movement of the actuator. The actuators 225 are movable within a predetermined range along the movement support 224. Referring to FIG. 5, the movement support 224 has a curved shape, and the plurality of ultrasound generating elements 222-1 through 222-n may be mounted on the curved movement support 224 so that they are movable within a predetermined range along the movement support 224.

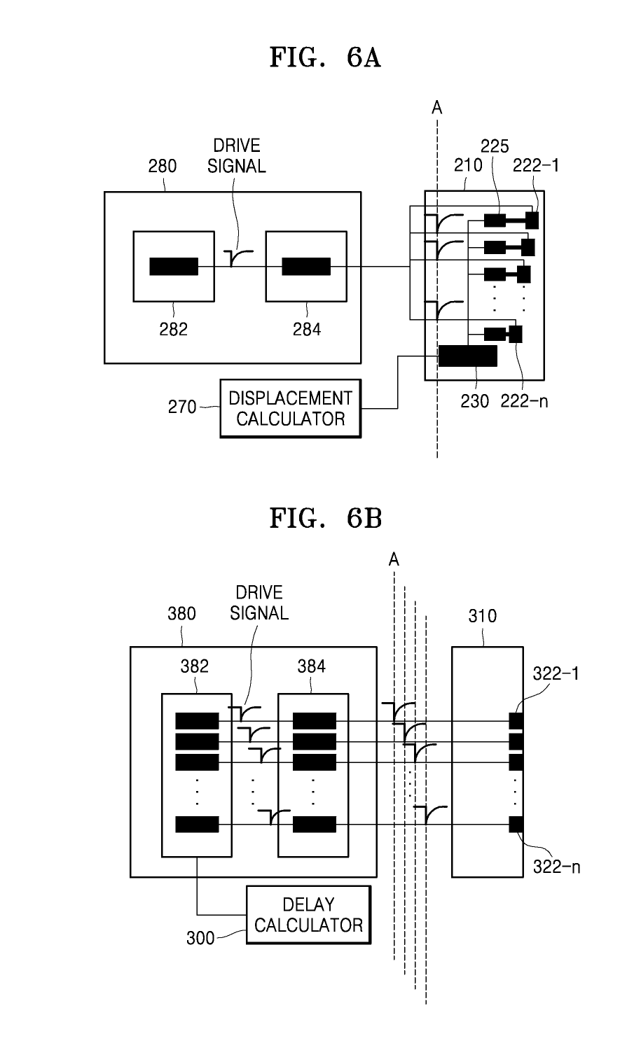

FIGS. 6A and 6B are diagrams illustrating examples of characteristics of an internal construction of a driver used in an ultrasound apparatus. More specifically, FIG. 6A illustrates an implementation of an ultrasound apparatus according to one or more embodiments, and FIG. 6B illustrates an example of an implementation of an ultrasound apparatus used in other technologies in the related art. Referring to FIG. 6A, an applicator 210 includes a plurality of ultrasound generating elements 222-1 through 222-n, a plurality of actuators 225, each being coupled to one side of a corresponding ultrasound generating element, and a displacement adjuster 230 for controlling the plurality of actuators 225. The displacement adjuster 230 receives calculated displacements of the individual ultrasound generating elements 222-1 through 222-n within the applicator 210 from a displacement calculator 270 and controls the actuators 225 corresponding to the individual ultrasound generating elements 222-1 through 222-n based on the received displacements. When the actuators 225 adjust the positions of the ultrasound generating elements 222-1 through 222-n, a driver 280 generates drive signals for driving the individual ultrasound generating elements 222-1 through 222-n simultaneously for transmission. As can be seen in FIG. 6A, the drive signals arrive at the same position at point in time ‘A’.

The ultrasound generating elements 222-1 through 222-n are driven in a particular order by adjusting the positions of ultrasound generating elements 222-1 through 222-n within the applicator 210. Thus, it is not necessary to vary the time for transmission of each of the drive signals input to the ultrasound generating elements 222-1 through 222-n. Accordingly, as shown in FIG. 6A, the driver 280 may be implemented as a single channel architecture including a single generator 282 and a power amplifier 284.

Referring to FIG. 6B, an applicator 310 includes a plurality of ultrasound generating elements 322-1 through 322-n, the positions of which cannot be adjusted within the applicator 310. To drive the ultrasound generating elements 322-1 through 322-n in a particular order, each drive signal has to be delayed and input to a corresponding ultrasound generating element, 322-1 through 322-n. As apparent from FIG. 6B, drive signals are generated at different times, delayed, and input to the respective ultrasound generating elements 322-1 through 322-n. Furthermore, the drive signals for driving the respective ultrasound generating elements 322-1 through 322-n are all at different positions at point in time ‘A’.

In this configuration, a delay calculator 300 is needed to calculate a delay value between drive signals input to the respective ultrasound generating elements 322-1 through 322-n. As shown in FIG. 6B, a driver 380 includes a plurality of power amplifiers 384 and a plurality of signal generators 382 for generating drive signals which are input to the respective ultrasound generating elements 322-1 through 322-n. In other words, since the driver 380 is implemented to have a multiple channel architecture, the driver 380 may have a complicated configuration different from the driver 280 shown in FIG. 6A.

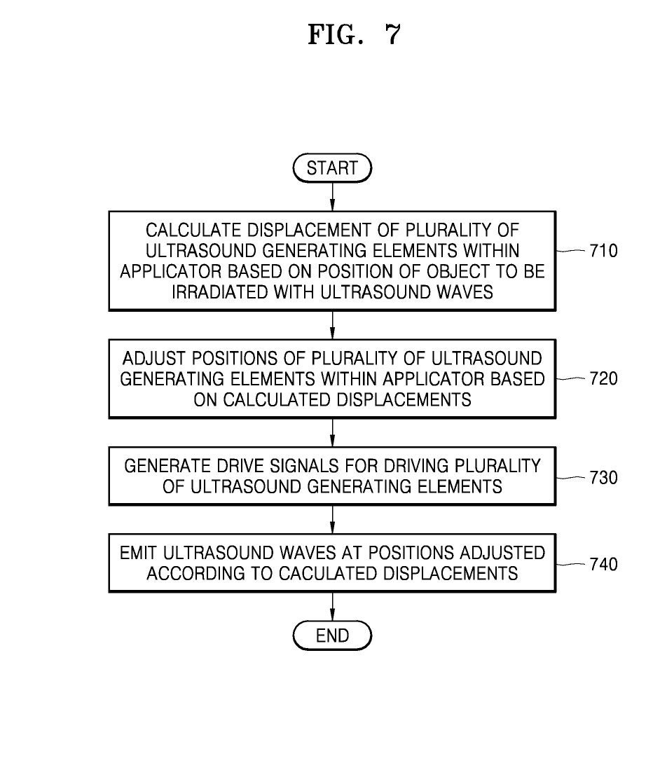

FIG. 7 is a diagram illustrating an example of a method of transmitting ultrasound. The operations in FIG. 7 may be performed in the sequence and manner as shown, although the order of some operations may be changed or some of the operations omitted without departing from the spirit and scope of the illustrative examples described. Many of the operations shown in FIG. 7 may be performed in parallel or concurrently. The above descriptions of FIGS. 1-6 with respect to an ultrasound system and an ultrasound apparatus is also applicable to FIG. 7, and thus will not be repeated here.

Referring to FIG. 7, in 710, displacements of a plurality of ultrasound generating elements 222-1 through 222-n, which comprise a transducer 220 within an applicator 210, are calculated based on the position of an object to be irradiated with ultrasound waves. The displacement of each ultrasound generating element is less than one wavelength of an ultrasound wave emitted thereby.

In 720, the positions of the plurality of ultrasound generating elements 222-1 through 222-n within the applicator 210 are adjusted based on the displacements. A location of a focal point where ultrasound waves are focused is determined by the positions of the ultrasound generating elements 222-1 through 222-n within the applicator 210. The direction of an ultrasound beam generated by the transducer 220 is steered by adjusting the positions of the ultrasound generating elements 222-1 through 222-n within the applicator 210. In 730, drive signals for driving the plurality of ultrasound generating elements 222-1 through 222-n are generated simultaneously.

In 740, the plurality of ultrasound generating elements 222-1 through 222-n emit ultrasound waves at positions adjusted according to the displacements. Each ultrasound wave emitted by a corresponding one of the ultrasound generating elements 222-1 through 222-n has a different phase value due to a difference in positions of the ultrasound generating elements 222-1 through 222-n within the applicator 210.

The methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An ultrasound apparatus comprising:
a transducer comprising a plurality of ultrasound generating elements controlled independent of each other and configured to emit ultrasound waves at positions adjusted according to calculated displacements;
a plurality of actuators configured to adjust position of the plurality of ultrasound generating elements;
a displacement calculator configured to calculate displacements of the plurality of ultrasound generating elements based on a position of an object to be irradiated with ultrasound waves;
a displacement adjuster configured to control the plurality of actuators based on the calculated displacements; and
a driver configured to drive the plurality of ultrasound generating elements.

2. The apparatus of claim 1, wherein the driver is further configured to simultaneously generate drive signals for driving the plurality of ultrasound generating elements.

3. The apparatus of claim 1, wherein a focal point where the emitted ultrasound waves are focused is determined by the positions of plurality of ultrasound generating elements.

4. The apparatus of claim 1, wherein each of the ultrasound waves emitted by the plurality of ultrasound generating elements has a different phase value due to a different position of each of the plurality of ultrasound generating elements.

5. The apparatus of claim 1, wherein the direction of an ultrasound beam generated by the transducer is changed by adjusting the positions of the plurality of ultrasound generating elements.

6. The apparatus of claim 1, wherein the calculated displacement is less than one wavelength of the emitted ultrasound wave.

7. The apparatus of claim 1, further comprising at least one movement support that is configured to support at least one of the plurality of actuators and to assist in movement of the at least one of the plurality of actuators, and
a movement controller configured to move the at least one actuator within a predetermined range along the at least one movement support.

8. The apparatus of claim 7, wherein, if the at least one actuator is moved along the at least one movement support, the displacement calculator is further configured to calculate the displacements of the plurality of ultrasound generating elements within the applicator based on positions of the plurality of ultrasound generating elements that are moved together with the at least one actuator.

9. The apparatus of claim 1, wherein the transducer, the plurality of actuators, and the displacement adjuster are disposed in an applicator.

10. The apparatus of claim 1, wherein the driver is implemented in a single-channel architecture, and the driver comprises a generator and a power amplifier.

11. A method of transmitting ultrasound waves using a transducer disposed within an applicator, the transducer comprising a plurality of ultrasound generating elements controlled independently of each other through a plurality of actuators, the method comprising:
calculating displacements of the plurality of ultrasound generating elements based on a position of an object to be irradiated with ultrasound waves;
adjusting positions of the plurality of actuators based on the calculated displacements;
generating drive signals for driving the plurality of ultrasound generating elements; and
emitting the ultrasound waves at the positions adjusted according to the calculated displacements.

12. The method of claim 11, wherein the generating of the drive signals comprises generating the drive signals simultaneously.

13. The method of claim 11, wherein a focal point where the emitted ultrasound waves are focused is determined by the positions of plurality of ultrasound generating elements.

14. The method of claim 11, wherein each of the ultrasound waves emitted by the plurality of ultrasound generating elements has a different phase value due to a different position of each of the plurality of ultrasound generating elements.

15. The method of claim 11, wherein the direction of an ultrasound beam generated by the transducer is changed by adjusting the positions of the plurality of ultrasound generating elements.

16. The method of claim 11, wherein the calculated displacement is less than one wavelength of the emitted ultrasound wave.

17. A non-transitory computer-readable recording medium having recorded thereon a program for executing the method of claim 11.

18. An ultrasound applicator comprising:
a housing with an open end;
a membrane disposed on the open end of the applicator and the membrane configured to contact the skin of a subject;
a transducer comprising a plurality of ultrasound generating elements controlled independent of each other, the transducer is disposed in the housing;
a plurality of actuators configured to adjust position of the ultrasound generating elements, the actuators are disposed in the housing; and
a displacement adjuster configured to control the plurality of actuators based on a position of the object to be irradiated with ultrasound waves, the displacement adjuster is disposed in the housing.

19. The apparatus of claim 18, wherein the transducer is disposed proximal to the membrane, the displacement adjuster is disposed proximal to a closed end of the housing, and the actuators are disposed between the ultrasound generating elements and the displacement adjuster.

20. The apparatus of claim 18, further comprising at least one movement support disposed in the housing, the movement support is configured to support at least one of the plurality of actuators and to assist in movement of the at least one of the plurality of actuators.

21. The apparatus of claim 20, wherein the shape of the movement support is linear, curved, or spiral.
